(19) United States
(12) Patent Application Publication
(10) Pub. No.: US 2016/0150981 A1
(43) Pub. Date: Jun. 2, 2016

(54) MONITORING SYSTEMS

(71) Applicant: CAMBRIDGE DESIGN PARTNERSHIP LLP, Cambridge Cambridgeshire (GB)

(72) Inventors: James George BAKER, Cambridge (GB); Lucy Ann SHELDON, Cambridge (GB); Ben James CRUNDWELL, Hertfordshire (GB)

(21) Appl. No.: 14/905,010

(22) PCT Filed: Jul. 14, 2014
(86) PCT No.: PCT/GB2014/052140

§ 371 Date: Jan. 14, 2016

(30) Foreign Application Priority Data
Jul. 15, 2013 (GB) 1312630.5

(51) Int. Cl. A61B 5/0205; A61B 5/00

(52) U.S. Cl. CPC A61B 5/02055; A61B 5/682; A61B 5/6838; A61B 5/6819; A61B 2562/029

(57) ABSTRACT

We describe a medical device, in embodiments a battlefield triage device, configured to be attached to and carried by a person. The device comprises a sensor module to attach to a person's face. The module having a respiratory sensing region comprising an air temperature sensor and a humidity sensor arranged such that, when the module is attached, airflow in and out of the person's lungs flows over said respiratory sensing region. The device also includes a signal processing system, coupled to the sensor module, to process and combine signals from the temperature sensor and from the humidity sensor to determine a respiration rate for the person and a system to output data indicating the respiration rate. The sensor is arranged to attach to/over the person's nose and/or mouth such that airflow in and out of the person's lungs, in particular, via the nostrils flows over the respiratory sensing region.

### FIELD OF THE INVENTION

This invention relates to devices and methods for detecting and monitoring heart rate and breathing, for example in battlefield environments.

### BACKGROUND TO THE INVENTION

The battlefield environment presents special problems for dealing with casualties. Triage is essential to identify the cohort of casualties who will benefit most from the correct medical treatment. Patient physiological monitoring is available for civilian settings but suitable equipment is not yet available for the battlefield. The battlefield itself is a noisy, dirty and chaotic environment and, more generally, can be subjected to extremes of temperature, humidity and the like.

Interviews with medical and other military personnel have identified a number of unmet needs including the need for fast, reliable measurement of respiratory rate, real time monitoring and display of vital signs, and access to vital signs trend data over time.

### SUMMARY OF THE INVENTION

According to a first aspect the invention provided a device, in particular a medical device such as a battlefield triage device, configured to be attached to and carried by a person, in particular a patient/casualty, the device comprising: an attachable sensor module, to attach to or in the vicinity of a person's face, the module having a respiratory sensing region comprising an air temperature sensor and a humidity sensor arranged such that, when the module is attached, airflow in and out of the person's lungs flows over said respiratory sensing region, the module further bearing an optical heart sensor arranged, when the module is attached, derive to an optical heart rate sensing signal from flesh of the person; a signal processing system, coupled to said sensor module, to process and combine signals from said temperature sensor and said humidity sensor to determine a respiration rate for said person and to process signals from said optical heart rate sensor to determine a heart rate of said person; a data output system, coupled to said signal processing system, to output data indicating one or both of said respiration rate and said heart rate.

Embodiments of the device are small, light, very quick to attach and may be cheap enough to be disposable. In some preferred embodiments the sensor is arranged to attach to the person's nose and/or mouth—tests have shown good results for both. In one embodiment the sensor module is configured to clip onto the person. It may thus have a pair of jaws, in particular sprung jaws, or other means to clip partially within a nostril (although it may additionally or alternatively be clipped onto the mouth). The respiratory sensing region is arranged such that, when the module is clipped or otherwise attached partially within the nostril, airflow in and out of the person's lungs via the nostril flows over the respiratory sensing region. Other attachment methods which may be employed for this arrangement include a sticker, over-centre/cam clip, and a use-once ratchet system (akin to a cable tie). In still other approaches the sensor may be carried by another item attached to the person/patient/casualty, such as an oxygen mask. The general objective is to locate the sensing region in the vicinity of the mouth or nose.

Clipping the sensor onto the nose allows the respiratory rate to be monitored accurately without significantly impeding airflow. Preferably the respiratory sensing region is located at the base of one of the jaws adjacent the articulating (hinging) region so that it sits just outside the nostril in use. This allows the sensor also to respond to a degree to airflow through the mouth (and, if necessary, this device configuration also allows the device to be clipped onto a lip rather than a nostril). Preferably the optical heart rate sensor mounted on the outer jaw, to avoid obstructing airflow within the nostril. Preferably this sensor is shielded from external sunlight (which may be very bright) by an opaque region of the jaw and/or a dedicated light shield. Alternatively light transmission through the flesh of the nose may be employed with a light source on one of the jaws and a light detector on the other, preferably the internal jaw. This works better but the portion of the sensor inside the nostril needs to be relatively small.

Thus in embodiments the sensor module comprises a single, clip-on unit which senses both temperature and humidity or the respiration rate and which includes an optical sensor for the heart rate. As explained in more detail below, sensing both temperature and humidity is important for reliable operation in a wide range of environments—for example a jungle environment may be extremely humid with little change in humidity between air flowing into the lungs and air flowing out of the lungs, and in such a situation the temperature change may be relied upon to determine the respiration rate.

As previously mentioned, in some preferred embodiments at least the sensor module is cheap, light and disposable. A medical officer can be reluctant to leave kit with a casualty because they may need it for others and thus it is preferable for the device to be potentially issued to each soldier (or other user) individually, for example as part of a med-pouch. In embodiments, therefore, the sensor module is fabricated from moulded plastic and the jaws may be joined by a curved, resilient plastic connector acting as both a hinge and a spring. In principle the plastic sprung jaws/housing of the sensor module may therefore be one-piece mouldable.

In embodiments of the device the sensor module may also incorporate the signal processing system and data output system in a single, self-contained unit which simply clips onto the person's/patient's/casualty's nose. In such an arrangement a display may be provided on the outer most jaw, that is the jaw on the outside of the nostril when the device is attached. In other configurations a separate processing module, preferably a clip-on or strap-on module is provided incorporating the signal processing system and data output system. With such an arrangement the signal processing system may be automatically activated (switched on or woken from a sleep state) by connecting the sensor module to the processing module. The connection may be wired or wireless although automatic switch on is more straightforward with a wired connection since physical connection can activate the processing module.

The data output system preferably comprises a display of both the respiration rate and heart rate of the person/patient/casualty, although a visual and/or audible alert may additionally or alternatively be provided responsive to detection of a deteriorating respiration and/or heart rate (for example less/greater than a threshold and/or less/greater than a threshold change over time). The display may be an (organic) light emitting diode display which is advantageous because it is physically thin and power efficient, or an LCD or electronic paper (electrophoretic) display both of which are more easily visible under high ambient light conditions, the latter being also very thin and power efficient.

Further additionally or alternatively the data output system may include a wired or wireless communication system, for example PRR (personnel role radio) communications. Preferred embodiments of the system include a non-volatile memory configured to store a user-accessible log of historical data for the respiration rate and heart rate. Preferably this is incorporated into the signal processing system and travels with the person/patient/casualty although, potentially, it may be provided on a removable memory module. Again this data may be accessed either directly using the processing module or indirectly via wired or wireless communications including, for example, near field communications. Optionally the processing module may be provided with a system to enable a medic to record MIST (mechanism, injury, signs, treatment) data, again to follow the person/patient/casualty (it can be very difficult to communicate such information in a noisy helicopter were verbal communication is difficult or impossible). Such a system may comprise, for example, an analogue or digital recording system. Optionally the above-described device, and the methods/devices described later, may monitor additional vital signs, for example oxygen saturation (SpO2 or other measure) and/or blood pressure and/or skin temperature.

As previously mentioned preferred embodiments of the signal processing system combines signals from both the temperature sensor and humidity sensor to determine a respiration rate for the person/patient/casualty. In embodiments these may be combined with a weighting dependent on a measure of fidelity or quality of the respective signal, for example a signal-to-noise ratio of the signal. In embodiments a respiration rate is determined from each of the temperature sensor and humidity sensor and these are combined as a weighted average, weighted by a fidelity measure of the signals from which each rate is derived.

In one preferred signal processing technique a signal from one or both of the temperature sensor and the humidity sensor is differentiated to identify changes in temperature/humidity to establish a respiration rate. This is advantageous because of the wide temperature/humidity ranges over which the device may have to operate. The differentiated signal may then be averaged and thresholded to provide a digital signal whose pulse rate determines the respiration rate.

When processing the signal from the heart rate sensor preferably the signal is filtered to inhibit dicrotic noise (some cardiac injuries can produce a dicrotic pulse). In embodiments this may be achieved by inhibiting detection of a pulse/transition in the heart rate sensor signal until a threshold duration from a previous pulse has elapsed, preferably a predetermined fraction of a measured inter-beat interval.

The invention also provides a (preferably clip-on) sensor module as described above; and a signal processing module comprising a signal processing system as described above.

In a related aspect the invention a method of monitoring a person, for example a patient or casualty for battlefield triage, the method comprising; monitoring both heart rate and respiration rate of said person with a combined sensor module by: attaching said sensor module in or adjacent to a nostril or mouth of said person such that one or both of a temperature sensor and humidity sensor is located in an airflow in and out of the person's lungs via said nostril and/or mouth; monitoring a respiration rate of said person using said temperature and/or humidity sensor; and monitoring heart rate of said person using an optical heart rate sensor mounted on said sensor module.

As previously described, preferably in use the temperature/humidity sensors hang just underneath the nose and, preferably, the respiration rate is determined from a combination of signals from both the temperature sensor and humidity sensor. Alternatively, however the sensor may be attached to the mouth—some preferred embodiments can be attached in either position or elsewhere on the face in the vicinity of the nose or mouth. Preferably the sensor module is a clip-on sensor module.

In a further related aspect the invention provides a clip-on sensor module for a monitoring both heart rate and respiration rate of a person, the sensor module comprising one or both of a temperature sensor and humidity sensor arranged such that they are located in an airflow in and out of the person's lungs via said nostril when the module is clipped partially within said nostril; and an optical heart sensor arranged, when the module is clipped partially within said nostril, to derive an optical heart rate sensing signal from flesh of said nostril.

In use the module is attached to or held in the vicinity of the nose and/or mouth.

The invention contemplates employing a device/method/module as described above in scenarios other than a battlefield. Thus aspects and embodiments of the invention provide a medical device/method/module as described above which may be used for battlefield triage but also devices for use, for example, in a general hospital environment.

More particularly there is a general need for accurate electronic respiration rate measurement systems for activities such as triage, activity monitoring, health monitoring, breathing monitoring and the like, and tests have established that embodiments of the inventions are able to produce accurate breathing rate determinations.

Thus in a further aspect the invention provides a device configured to be attached to and carried by a person, the device comprising: a respiration rate sensor comprising an air temperature sensor and a humidity sensor; a signal processing system to process and combine signals from said temperature sensor and said humidity sensor to determine a respiration rate for said person; and a data output system, coupled to said signal processing system, to output data indicating said respiration rate for monitoring said person.

In some preferred embodiments the device further comprises a heart rate sensor, and the signal processing system is additionally configured to process signals from the heart rate sensor to determine a heart rate of the person.

The skilled person will appreciate that features of the above described aspects and embodiments of the invention may be combined in any permutation.

### BRIEF DESCRIPTION OF THE DRAWINGS

The invention is diagrammatically illustrated, by way of example, in the accompanying drawings in which:

FIGS. 1a and 1b illustrate a battlefield triage monitoring device according to alternative embodiments of the invention;

FIGS. 2a to 2e illustrate a method of using a battlefield triage monitoring device according to an embodiment of the invention;

FIG. 3 illustrates the functionalities of a nose clip of a battlefield triage device;

FIG. 4 shows a detailed view of a nose clip of a battlefield triage device;

FIG. 5 shows a monitoring device in use on a casualty;

FIG. 6 shows an exemplary display panel of a battlefield triage device;

FIGS. 7a to 7c illustrate a display device to be used in conjunction with a battlefield triage device according to an embodiment of the invention;

FIG. 8 shows an alternative connector for a display component of a battlefield triage device;

FIGS. 9a to 9c show alternative arrangements of the battlefield triage device;

FIGS. 10a to 10c show, respectively, a block diagram of a processing module and of a sensor module for a battlefield triage device according to an embodiment of the invention, and a block diagram of signal processing code operating on data from temperature and humidity sensors for the device;

FIGS. 11a and 11b show, respectively, a graph illustrating a variation of temperature variation with time measured by an embodiment of the device, and a differentiated/thresholded version of the signal of FIG. 11a; and

FIG. 12 shows illustrates operation of the respiratory rate sensing system of a battlefield triage device according to an embodiment of the invention.

In the drawings, the following reference numerals are used:

10=battlefield triage device

12=nose clip sensor module

13=hinge

14=connecting wire

16=latching connector

18=processing module with display device/interface

20=display

22=external battery

24=clip

26=power button

28=packaging

30=casualty (patient) nose

32=casualty's/patient's clothing

34=sound alarm

36=light alarm

38=medic's display device

40=latching connector

42=headband-based device

44=sucker-based device

46=combined display and sensor

48=respiration sensor region

50=heart monitor

51=light shield

52=light

54=air-flow

56=combined temperature/humidity sensor

58=trend graph

60=critical information

62=icons

64=personal role radio (“PRR”)

### DETAILED DESCRIPTION OF PREFERRED EMBODIMENTS

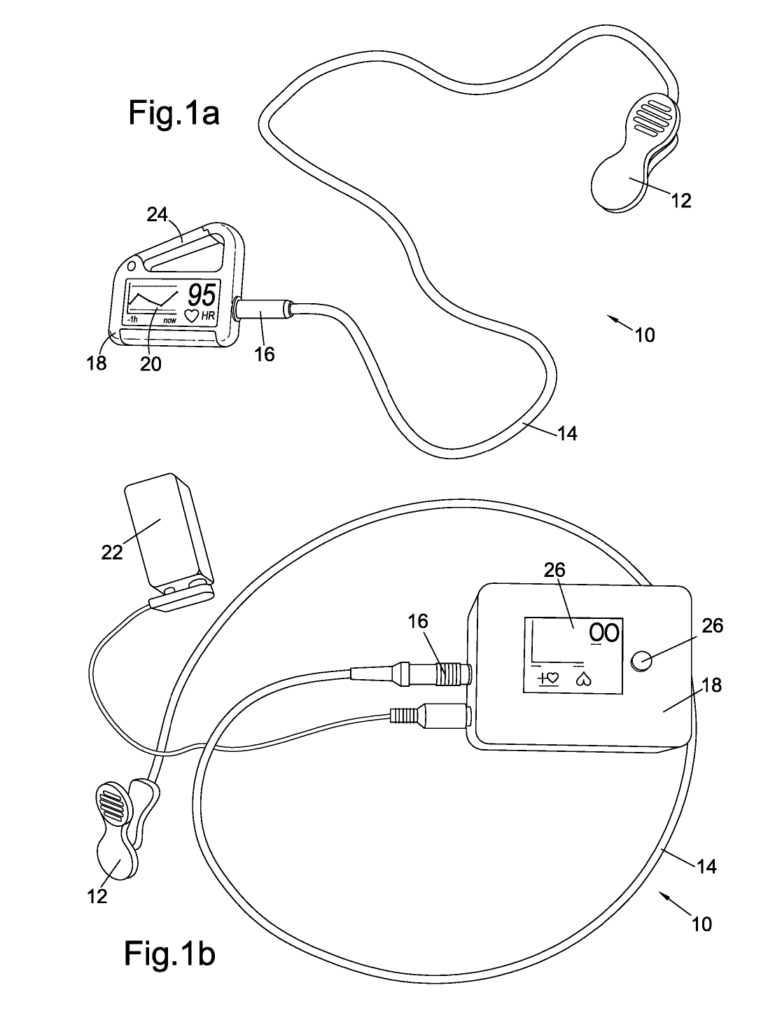

FIGS. 1a and 1b show examples embodiments of an integrated patient monitor battlefield triage device 10 according to the invention. Broadly speaking this comprises a sensor module in the form of a nose clip 12 and a processing module 18 with a display device. The device of FIG. 1b is a functional prototype. The monitoring device 10 is capable of providing:
Fast, reliable measurement of respiratory rate and heart rate—this is important because respiratory rate is considered the most important measure of patient criticality and current manual measurement methods are difficult, time consuming and inaccurate;
Real-time monitoring and display of vital signs—this overcomes the need for manual measurement, which is time consuming and needs to be repeated regularly; and
Access to vital signs trend data over time to medical personnel—this is crucial in an environment where front-line medics may not be able to regularly measure, or record and communicate their manual readings.
Alerts when vital signs fall out with health patient norms as defined in standardised triage processes and triage sieves. For example, when breathing rate is 30 breaths per minute or higher, or less than 10 breaths per minute.

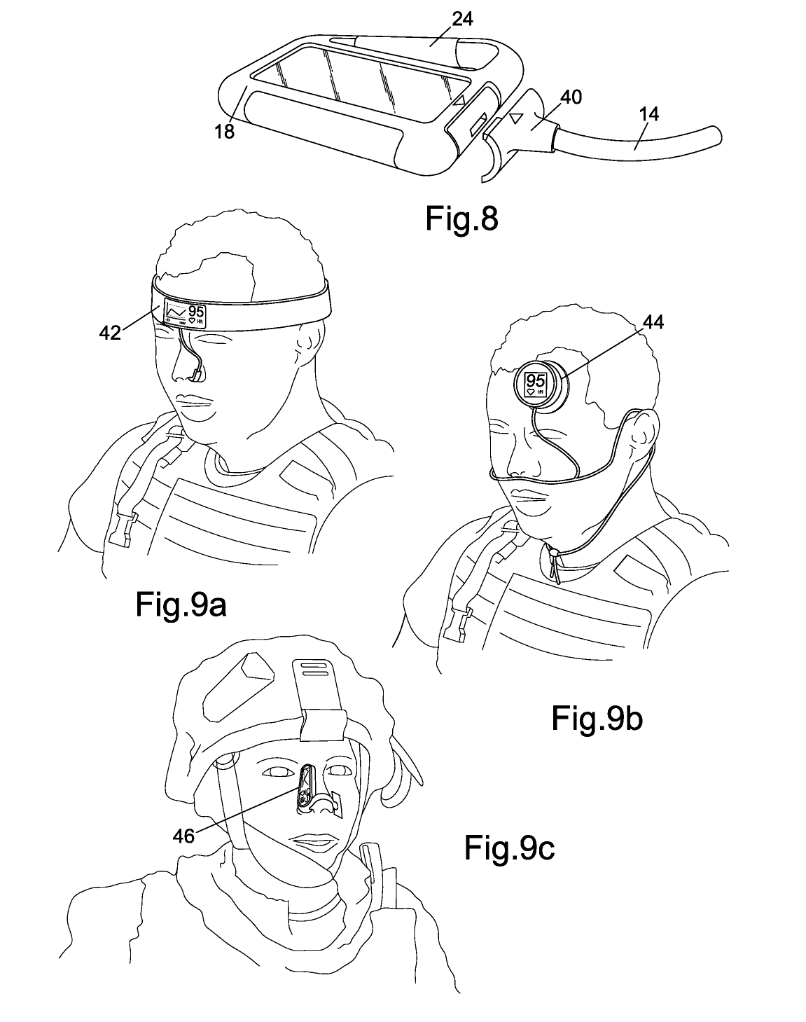

The nose clip 12 contains sensors to measure respiratory rate and heart rate. The nose clip may be disposable, which removes the risk of contamination transfer between patients. In the embodiment of the invention shown in FIGS. 1a and 1b, nose clip 12 is a sprung clip that is designed to attach to a patient's nose or ear. Such an attachment method allows the sensors to be quickly and easily attached to a patient. Nose clip 12 also comprises a connecting wire 14, which allows connection of the nose clip to the processing module 18. The connection of wire 14 to processing module 8 may, in embodiments, be made using a latching connector 16, which once connected, may also power-up the display 20 of the processing module 18. An alternative embodiment of the connector is depicted in FIG. 8, where custom latching connector 40 has a larger surface area and a shape that allows a user to grip the connector more easily. The size and shape of the connector 40 in combination with guide arrows on the connector and the processing module 18 allow a user to quickly connect the sensors to the processing module 18, particularly when in a stressful and/or low-light environment.

In FIG. 1a, the processing module 18 contains a battery (not visible) to power the device, and the device is powered-up automatically once the nose clip 12 is connected to the processing module 18 via the latching connector 16. FIG. 1b shows a prototype design in which a battery 22 is external to the processing module 18, however, it is preferable for the power source to be located within the display device. The processing module 18 of FIG. 1b comprises a power button 26 that needs to be depressed (i.e. is in an “on” position) in order to power up the display. In this embodiment, the processing module 18 may receive and store data even when the power button 26 is not depressed (i.e. is in an “off” position), thus saving the power associated with powering the display screen 26.

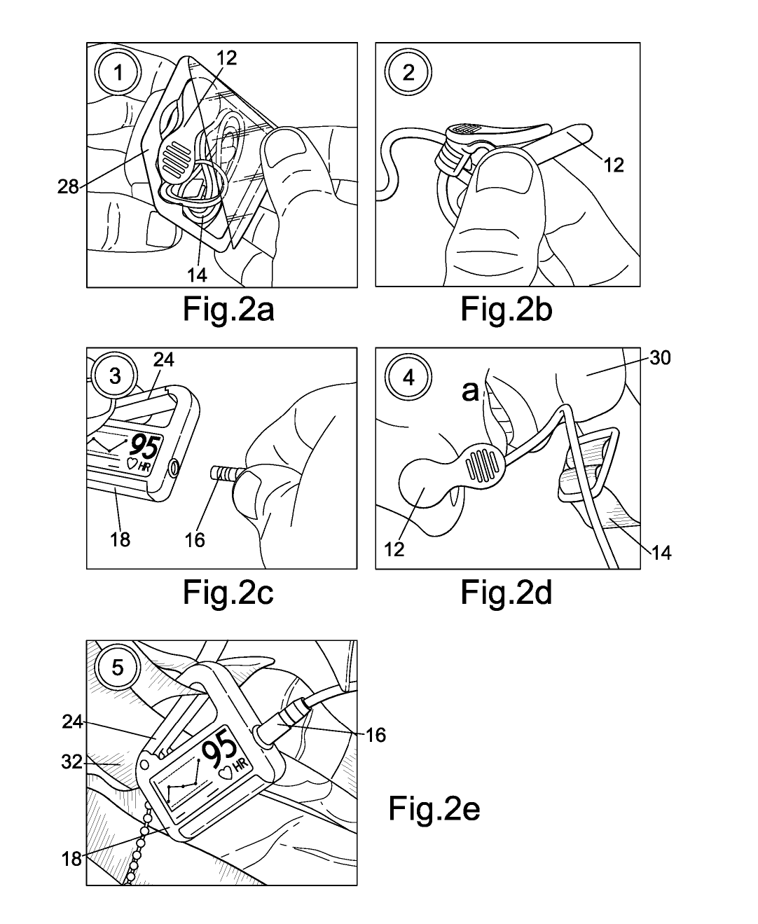

FIGS. 2a to 2e illustrate deployment of a battlefield triage according to an embodiment of the invention. Thus in FIG. 2a the device is packaged 28 in the form of a single use disposable sensor. The packaging is opened, the sensor is removed (FIG. 2b) and connected to the processing module by means of connector 16, automatically turning the module on (FIG. 2c). The sensor is clipped so that it lies partially within the nostril of the casualty (FIG. 2d), and then the processing module is attached by a clip 24 to a belt 32 on the casualty's clothing (FIG. 2e).

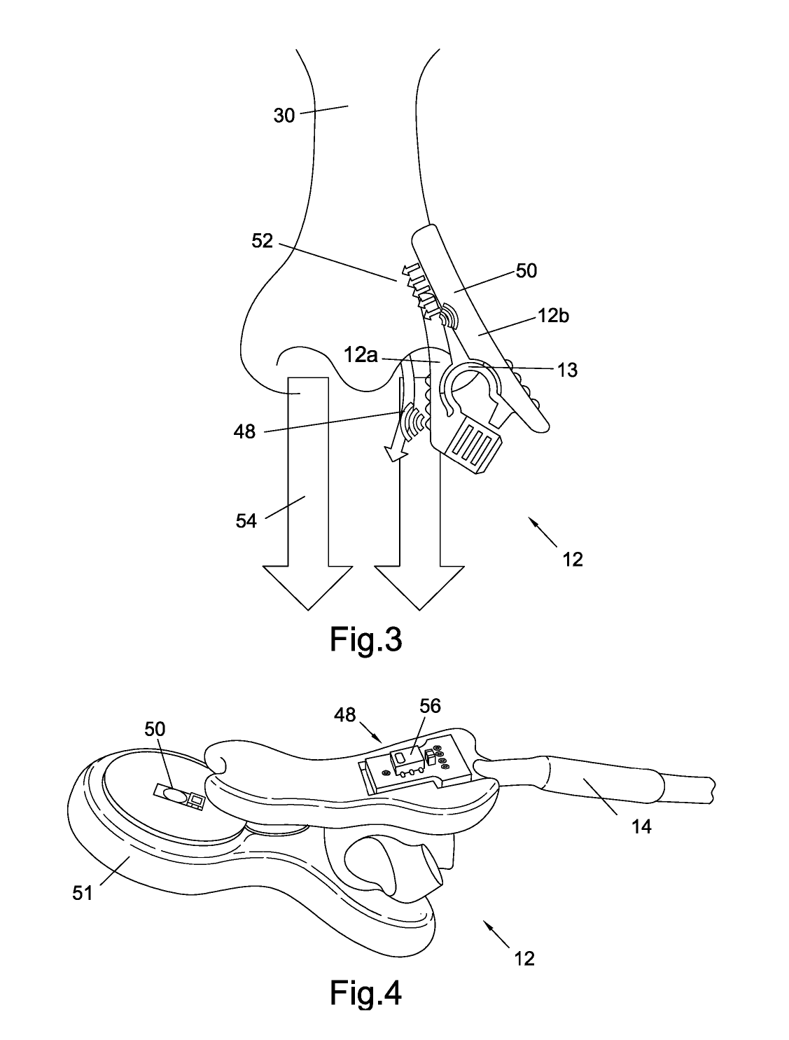

FIG. 3 shows the nose clip sensor module 12 attached so that it lies partially within the nostril 30 of the casualty's nose. The nose clip comprises inner 12a and outer 12b jaws hinged and resiliently biased together by a plastic spring 13. A respiratory sensor region 48 comprises temperature and humidity sensors (which may be combined in a single sensor package) and is located at the lower end of the inner jaw 12a, just outside the nostril and in the airflow into/out of the nostril. A heart rate sensing region 52 comprises an optical reflectance sensor mounted on the outer jaw 12b.

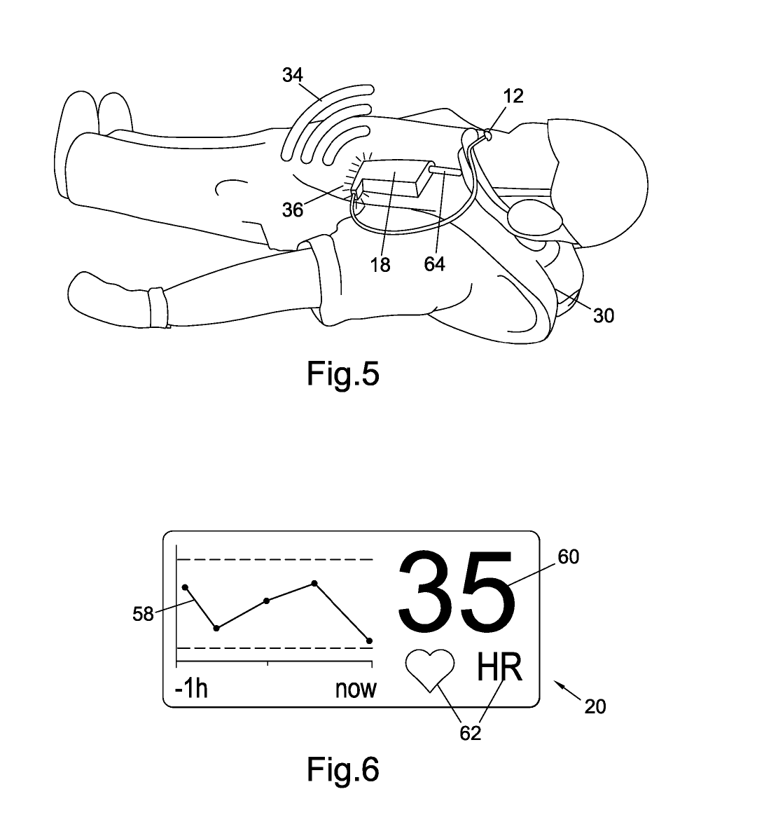

FIG. 4 shows a prototype embodiment of the sensor module comprising a combined temperature/humidity sensor 56, in a prototype an SHT21 sensor from Sensirion. In other, cheaper approaches separate humidity and temperature sensors may be employed, for example using a low cost humidity sensor such as the HCZ-D5-A from Multicomp. The skilled person will appreciate that there are many different ways of sensing humidity, including by detecting a change in resistance and/or capacitance of a sensing element. A temperature sensor for the module may comprise, for example a thermocouple.

As described in more detail below, the respiratory rate is measured by sensing differences in temperature and humidity between inhaled and exhaled air, converting this into a measurement of breaths per minute. By locating the sensor as illustrated in FIGS. 3 and 4, outside the nostril, the respiratory rate sensor is also able to respond to air exhaled from the mouth, improving the reliability of the measurement.

In embodiments the heart rate is detected by illuminating the flesh of the nose with light (which may be green light), for example from an LED, and sensing either the light transmitted through the nose or the level of light reflected back. The level of transmitted/reflected light is modulated according to blood flow into the nose. The nose has a good supply of subcutaneous blood vessels which alternately expand and contract in time with the heart rate, which can be detected as a small variation in the transmitted and/or reflected light. To avoid impeding the air flow within the nose preferably a reflectance sensor mounted on the outer jaw 12b is employed.

In the illustrated prototype the nose clip was fabricated from translucent plastic, which was coloured black 51 to shield the sensor from sunlight. The sensor was tested under a wide range of lighting conditions and reliability was reduced under high direct sunlight levels (90,000 lux). To address this sufficient shielding is employed for the sensor not to be saturated, optionally also arranging the readout electronics accordingly; an additional light shield (not shown) may also be employed.

FIG. 5 illustrates an embodiment of the battlefield triage sensor coupled to a PRR (personnel role radio) 64 used to transmit an alert to a medical officer in response to the processing module identification of deteriorating respiratory rate and/or heart rate indicators. In addition an audible and/or visual alert may be provided.

FIG. 6 illustrates an example display 20 of the processing module. As illustrated this shows an indication of a heart rate 60 with corresponding heart rate icons 62 and a trend graph 58 showing the changes in heart rate since the device was activated. A similar display may be provided for respiration rate.

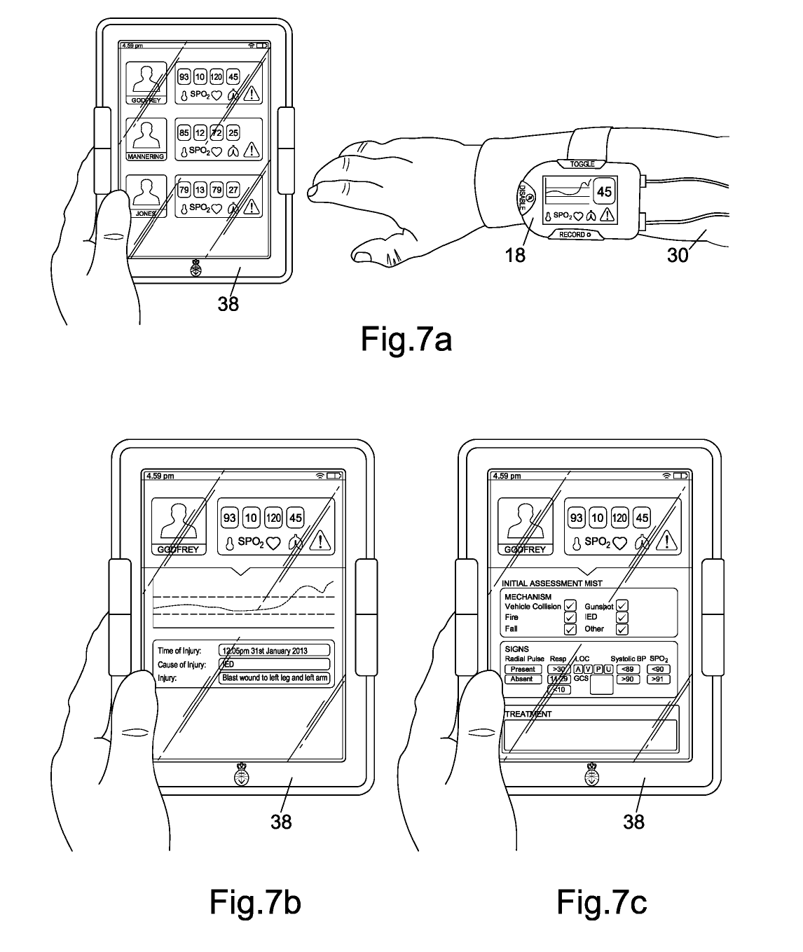

FIG. 7 illustrates examples of an additional display module 38 for use by a medic, coupleable to the processing module for example by a wired or wireless connection. The display 38 may provide additional information and/or functionality, for example to facilitate recording and replay/transfer of additional information such as MIST information in conjunction with the casualty data, for later use. Thus the module 38 may include an analogue or a digital recording system and means for extracting the recorded data.

FIG. 8 illustrates a preferred, latching connector 40 for connecting the sensor and processing modules. The illustrated connector has mating components which facilitate connector orientation under stress/in low lighting conditions (a long axis of the connector head aligning parallel to an edge of the processing module).

FIG. 9a illustrates a system in which the processing module is mounted on a head band 42. FIG. 9b illustrates a system in which the processing module is attached to the casualty by a sucker 44. FIG. 9c illustrates a device in which the processing system is incorporated into the sensor module making a single self-contained device 46. This removes the need for a cable and facilitates in-field deployment as well as helping to ensure that the display is visible, even on a moving casualty.

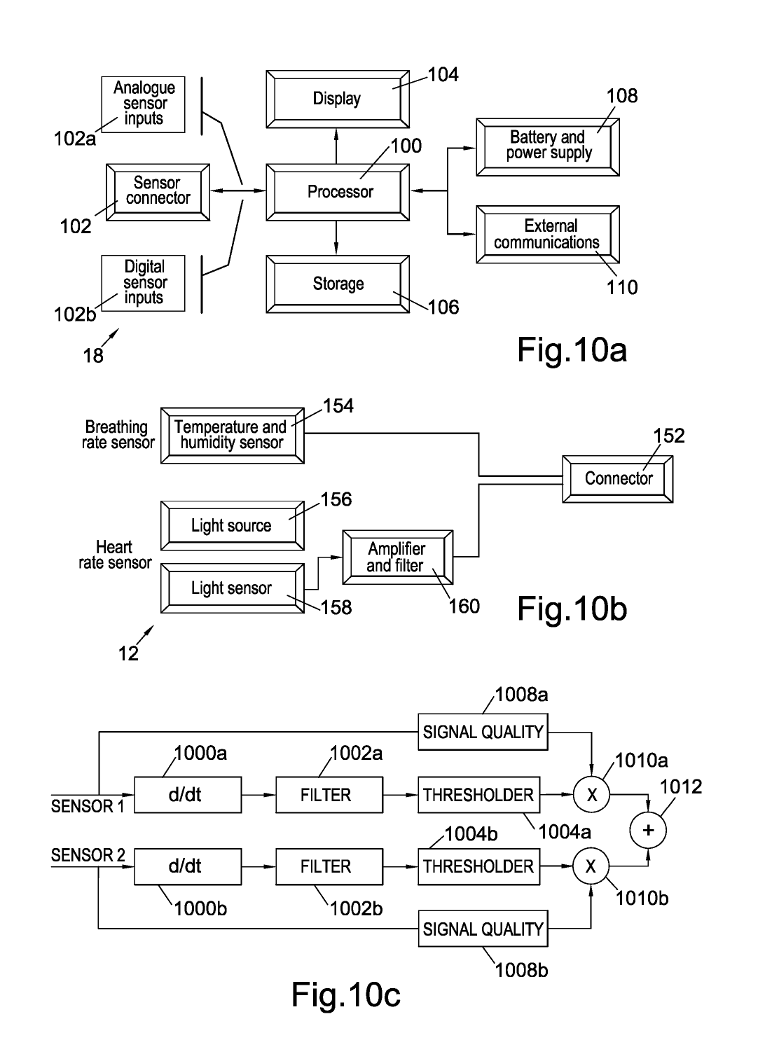

Referring next to FIG. 10, FIG. 10a shows a functional block diagram of the processing module 18. This comprises a microprocessor 100 coupled to a sensor connection 102 which provides connections for analogue sensor inputs 102a and digital sensor input 102b, as well as power for the sensors. Processor 100 drives a display 104, for example an OLED or LCD display, and is coupled to storage 106 which comprises non-volatile processor control code storage for the operating system, user interface and signal processing algorithms as well as other functions such as communications, non-volatile data memory storage for logging sensor data, and working memory. The processor 100 is also coupled to a battery and power supply module 108; this may either be switched on by sensor connector 102 when the sensing module is connected, or sensor connector 102 may be employed to wake up processor 100. An external communications module 110 provides wireless communications for downloading data from storage 106.

FIG. 10b shows a functional block diagram of the sensor module 12. This comprises a connector 152 to mate with connector 102 on the processing module to provide analogue and digital connections to the breathing rate and heart rate sensors. Connector 152 connects to temperature and humidity sensors 154 providing a breathing rate sensor, in one embodiment an SHT21 from Sensirion AG, which provides a digital I²C digital interface; in other embodiments separate temperature and humidity sensors. Connector 152 also provides power to a light source 156 (as well as to the sensors) and is connected to a light sensor 158 such as an APDS9008 from Avago Technologies, which is in turn coupled to an amplifier and filter 160 providing an analogue signal to connector 152 and processor 100.

Processor control code running on processor 100 reads and calculates sensor values, logs data to storage 106, drives the display 104, and allows the logged data to be downloaded via communications 110. The software provides a common framework for monitoring raw sensor data; in embodiments the sensor values are updated at 500 Hz, which allows noise to be rejected and improves sensor accuracy. Sensor algorithms detect fluctuations in the raw sensor data to identify a regular pattern, identifying and timing peaks to calculate the rate of the measured parameter, which is then displayed/stored/otherwise processed.

For the heart rate sensing chain, the maximum and minimum sensor signals are recorded and a threshold is set midway between the two. The software then monitors the point at which the recorded signal crosses this threshold. This value is then checked to determine that it lies within physically realistic limits and is therefore a genuine measurement (noise reduction) and the value is also filtered to suppress dicrotic noise, in embodiments by delaying by 0.6 of the previous inter-beat interval. The inter-beat interval is then calculated by determining the period of time which has lapsed between pulses, and this is then fed into a ten point rolling average filter to suppress the effect of false readings. The filtered inter-beat interval is then used as the heart rate variable for the subsequent processing.

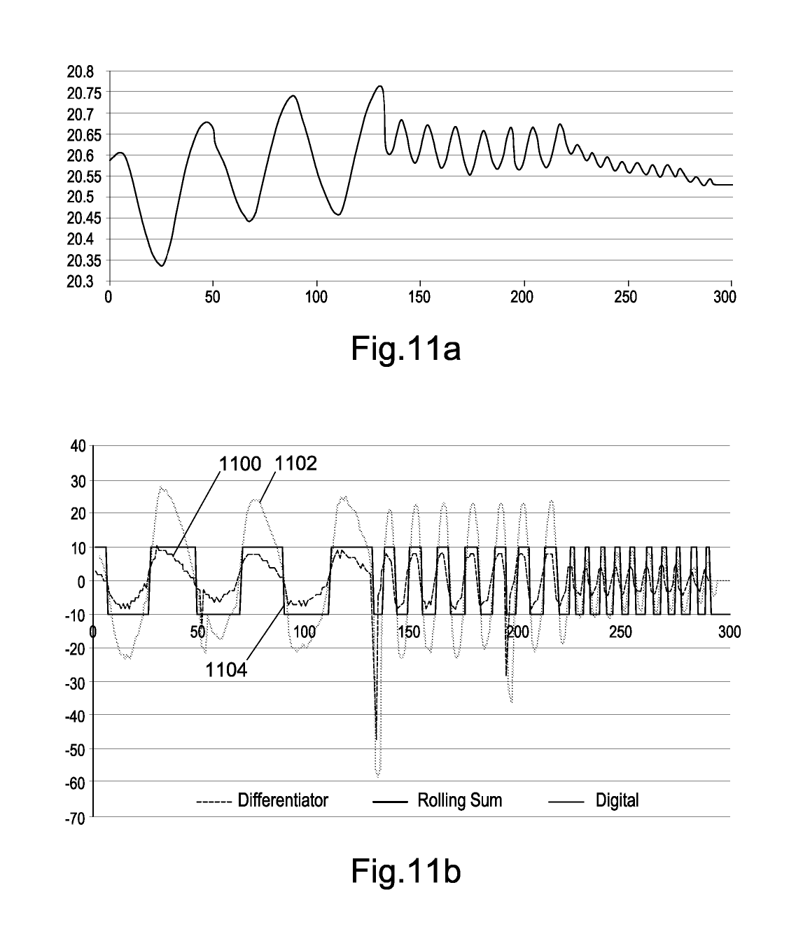

FIG. 11a shows a graph of temperature variation against time (arbitrary units), showing detection by the sensor of small changes in temperature caused by heating from warm breath and subsequent cooling from evaporation of moisture. These effects create a regular, approximately sinusoidal signal, which can be analysed to calculate the respiratory rate. As can be seen in FIG. 11a, the temperature changes over time, in part because the exhaled air warms up the sensor. Thus when processing the temperature sensor data the raw temperature readings are first differentiated to determine a rate of change of temperature and this differentiated signal is then fed into a three-point rolling sum, which averages the signals without requiring floating point maths. This signal is then converted to a digital signal by placing a threshold at zero with a hysteresis of plus/minus 1. FIG. 11b shows the differentiated signal 1100 from FIG. 11a, the rolling sum 1102, and the digital signal 1104 which results. This digital signal is then available to the system as a respiratory rate variable. The data from the humidity sensor can be treated in a similar manner.

Referring to FIG. 10c this shows a block diagram of the signal processing code operating on data from both temperature and humidity sensors. In one approach a selection may be made between the data from one or other sensor depending upon the quality of the data, for example the signal to noise ratio, amplitude of the signal or differentiated signal, and the like. In another approach, however, as illustrated, a respiratory rate variable derived from each sensor is combined weighted by a quality of the respiratory rate measurement as determined from the sensor data, for example by a signal to noise ratio measurement of the raw sensor data. Thus the arrangement of FIG. 10c illustrates, for each sensor path, a differentiating unit 1000a, b followed be a filter 1002a, b followed by a thresholder 1004a, b, providing respective respiratory rate outputs 1006a, b. A signal quality measuring module 1008a, b may be used to measure a quality of the raw sensor data, for example determining a signal to noise ratio or an amplitude of the signal or by making some other quality measure. The respective signal quality measures may then be then used to determine a proportion of each respiratory rate variable contributing to an overall determined respiratory rate via respective multipliers 1010a, b and a summer 1012, to sum the respiratory rates in proportion to their quality. The overall output may be scaled appropriately (not shown) and/or calibrated as needed.

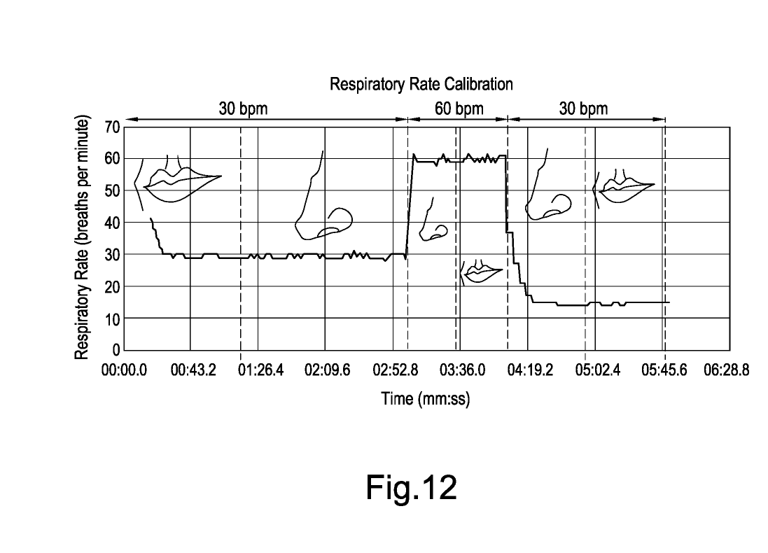

FIG. 12 illustrates operation of the respiratory rate sensing system, with an initial calibration performed by a patient artificially controlling their breathing rate. The mouth and nose icons in FIG. 12 illustrate detection of breathing rate via the nose and mouth respectively; it can be seen that the breathing rates match. Other experiments (not shown) demonstrated that wind had little effect on the measured breathing rate.

As previously described, additional functions performed by the processing module 18 include logging data to the internal memory and updating the display, for example every second. In one embodiment the onscreen history graph comprised 30 values and each time the buffer filled the data was compressed in half (and smoothed) and the time base of the graph increased. With this approach only 30 data points needed to be stored, substantially reducing the amount of memory required. To download data a button may be pressed on the unit or the external communications 110 may automatically connect, for example via nearfield coupling. A serial interface may be provided to access the stored data via one or both of a wired and wireless link. Regular or continuous monitoring may be provided if desired.

Although we have described example embodiments and applications of a medical/battlefield device for monitoring casualties for battlefield triage it will be appreciated that applications of the technology are not limited to this scenario. Thus the technology we have described is also useful in a hospital or ambulance environment. More generally applications may also include, for example, activity monitoring, health monitoring, breathing monitoring and the like. Still more generally, the technology may even be applied to suitable animals as well to people.

Thus in embodiments the invention also provides a device as described previously according to aspects/embodiments of the invention for use with non-patients, for example for: fitness purposes, recreational purposes, health monitoring purposes, wellness monitoring purposes, anxiety monitoring purposes, monitoring respiration rate during the practice of sport/activity/yoga and the like, managing stress and anxiety, and so forth. In further embodiments the invention provides a device to provides an alert when one or more vital signs deteriorates beyond a healthy norm, for example a norm in line with triage and/or medical procedures. Broadly speaking embodiments the invention have applications whenever monitoring breathing rate and heart rate using a sensor in the vicinity of the face can be employed. The sensor may be attached directly using clips, stickers and the like, or emplaced within another product which holds it in the correct vicinity, such as an oxygen supply mask.

No doubt many other effective alternatives will occur to the skilled person. It will be understood that the invention is not limited to the described embodiments and encompasses modifications apparent to those skilled in the art lying within the spirit and scope of the claims appended hereto.

1. A device configured to be attached to and carried by a person, the device comprising:
an attachable sensor module, to attach to or in the vicinity of a person's face, the module having a respiratory sensing region comprising an air temperature sensor and a humidity sensor arranged such that, when the module is attached, airflow in and out of the person's lungs flows over said respiratory sensing region;
a signal processing system, coupled to said sensor module, to process and combine signals from said temperature sensor and said humidity sensor to determine a respiration rate for said person;
a data output system, coupled to said signal processing system, to output data indicating said respiration rate.

2. A device as claimed in claim 1, the module further bearing an optical heart sensor arranged, when the module is attached, to derive an optical heart rate sensing signal from flesh of the person, wherein said signal processing system is further configured to process signals from said optical heart rate sensor to determine a heart rate of said person, and wherein said sensor module is configured to attach to or over a nostril of the person such that when the module is attached airflow in and out of the person's lungs via said nostril flows over said respiratory sensing region and such that said optical heart rate sensing signal is derived from flesh where the module is attached.

3. A device as claimed in claim 2 wherein said sensor module is a clip-on sensor module.

4. A device as claimed in claim 3, wherein said sensor module comprises a pair of jaws to clip the module to or over said nostril and wherein said respiratory sensing region is located outside said nostril when the module is clipped to or over said nostril.

5. (canceled)

6. A device as claimed in claim 3 wherein said optical heart rate sensor comprises a reflectance sensor located outside said nostril and shielded from external light when the module is clipped to or over said nostril.

7.-8. (canceled)

9. A device as claimed in claim 1 further comprising clip-on or strap-on processing module housing said signal processing system and said output system.

10. A device as claimed in claim 9 wherein said signal processing system is configured to be activated by connecting said sensor module to said processing module.

11. A device as claimed in claim 1 wherein said signal processing system and data output system are contained within said sensor module to provide a one-piece, self-contained monitoring device.

12. A device as claimed in claim 2 wherein said data output system comprises a display of said respiration rate and said heart rate, and wherein said signal processing system further comprises a user-accessible log of historical data for said respiration rate and said heart rate.

13.-15. (canceled)

16. A device as claimed in claim 2 wherein said signal processing system is configured to filter a signal from said optical heart rate sensor to inhibit dicrotic noise.

17. A device as claimed in claim 1 wherein the device is a battlefield triage device and wherein the person is a casualty.

18.-19. (canceled)

20. A method of monitoring respiration rate of a person, the method comprising:
attaching a sensor module in or adjacent to a nostril or mouth of said person such each of a temperature sensor and a humidity sensor is located in an airflow in and out of the person's lungs via said nostril and/or mouth; and
monitoring a respiration rate of said person using both said temperature and said humidity sensor; and
determining said respiration rate from a combination of signals from said temperature sensor and said humidity sensor.

21.-22. (canceled)

23. A device configured to be attached to and carried by a person, the device comprising:
a respiration rate sensor comprising an air temperature sensor and a humidity sensor;
a signal processing system to process and combine signals from said temperature sensor and said humidity sensor to determine a respiration rate for said person; and
a data output system, coupled to said signal processing system, to output data indicating said respiration rate for monitoring said person.

24. A device as claimed in claim 23 further comprising a heart rate sensor;
wherein said signal processing system is additionally configured to process signals from said heart rate sensor to determine a heart rate of said person; and wherein said data output system is additionally configured to output data indicating said heart rate for monitoring said person.

25. A device as claimed in claim 24 in the form of an attachable sensor module, to attach to a person's face, the module having a respiratory sensing region comprising said air temperature sensor and said humidity sensor and arranged such that, when the module is attached, airflow in and out of the person's lungs flows over said respiratory sensing region, the module further bearing an optical said heart sensor arranged, when the module is attached, derive to an optical heart rate sensing signal from flesh of the person.

26. A device as claimed in claim 25 wherein said sensor module is a sensor module configured to attach to or over a nostril of the person and arranged such that, when the module is attached partially within said nostril, airflow in and out of the person's lungs via said nostril flows over said respiratory sensing region and such that, when the module is attached to or over said nostril, said optical heart rate sensing signal is derived from flesh where the module is attached.

27. A device as claimed in claim 26 wherein said sensor module is a clip-on sensor module comprising a pair of jaws to clip the module into or over said nostril.

28. (canceled)

29. A device as claimed in claim 27 wherein said respiratory sensing region is located outside said nostril when the module is clipped to or over said nostril.

30. (canceled)

31. A device as claimed in claim 23 wherein said signal processing system is configured to determine a first respiration rate from said temperature sensor and a second respiration rate from said humidity sensor, and to combine said first and second respiration rates to determine said respiration rate for said person.

32. A device as claimed in claim 23 wherein said signal processing system is configured to filter a signal from said optical heart rate sensor to inhibit dicrotic noise.
